(19) United States
(12) Patent Application Publication
(10) Pub. No.: US 2004/0122356 A1
(43) Pub. Date: Jun. 24, 2004

(54) Kits and methods for the collection, storage and/or feeding of breast milk

(76) Inventors: Adam P. Burke, Williamsburg, VA (US); Randolph G. Gay III, Chesapeake, VA (US)

Correspondence Address: NIXON & VANDERHYE, PC, 1100 N GLEBE ROAD, 8TH FLOOR, ARLINGTON, VA, 22201-4714, US

(21) Appl. No.: 10/326,889

(22) Filed: Dec. 23, 2002

(51) Int. Cl. A61M 1/06

(52) U.S. Cl. 604/74

(57) ABSTRACT

Kits and methods for the collection, storage and/or feeding of breast milk include at least one flexible open-ended liner, an outer rigid tubular bottle adapted to receive the liner, and a breast pump adapter adapted to be threadably coupled to the bottle at one end and to a breast pump at another end thereof so as to allow breast milk to be expressed directly into the liner. A rigid stand for dependently supporting a flaccid liner containing breast milk, and at least one cap for sealably closing the open end of the liner may also be provided. The breast milk-containing liner may thus be sealed by the cap and positioned in the rigid stand. The stand and capped liner may then be placed in a refrigerated environment (e.g., a refrigerator, freezer, cooler or the like) so that the breast milk may be stored and preserved for future use. Most preferably, the kits of the present invention, will also include a nipple cap for the bottle. The chilled or frozen breast milk in the flaccid liner may thus be warmed and placed in the rigid tubular bottle so that the liner is dependently supported thereby. The nipple cap may then be threadably coupled to the bottle to allow the breast milk contents in the liner to be fed to the nursing infant.

### FIELD OF THE INVENTION

The present invention relates generally to the field of kits and methods by which breast milk may be collected, stored and/or subsequently used for infant feeding. In especially preferred embodiments, the present invention relates to kits and methods whereby open-ended flaccid liners having a self-supporting rim may be employed as a component part of a system for the common collection, storage and/or feeding means thereby avoiding unnecessary handling and/or dispensing of the breast milk.

### BACKGROUND AND SUMMARY OF THE INVENTION

A mother's breast milk is widely recognized as being significantly important to a nursing infant's nutrition and general health. A substantial problem to breast feeding, however, is that once the breast milk is expressed it must be used essentially immediately. The breast milk may, however, be frozen which increases its shelf life, but such a task usually entails the excessive handling of the breast milk requiring transfer from the collection container, to a freezing container, and then to the final feeding container. Unnecessary handling of the breast milk clearly is inconvenient to the mother and may therefore be one reason why breast milk is not more routinely collected for freezing.

It would therefore be highly desirable if kits and/or methods could be provided whereby breast milk could be collected, stored (especially frozen) and/or fed to a nursing infant in a more convenient manner. It is towards fulfilling such a need that the present invention is directed.

Broadly, the present invention is embodied in kits and/or methods and/or systems whereby breast milk may be collected, stored (especially frozen) and/or fed to a nursing infant in a more convenient manner. In especially preferred embodiments, the present invention includes open-ended flaccid liners having a self-supporting rim which may be employed as a component part of a system for the common collection, storage and/or feeding of breast milk. The unnecessary handling and/or dispensing of the breast milk may thereby be avoided.

Most preferably, the present invention is embodied in a breast milk collection kit which includes at least one flexible open-ended liner, an outer rigid tubular bottle adapted to receive the liner, and a breast pump adapter adapted to be threadably coupled to the bottle at one end and to a breast pump at another end thereof so as to allow breast milk to be expressed directly into the liner.

The present invention may also be embodied in a breast milk storage kit which further comprises a rigid stand for dependently supporting a flaccid liner containing breast milk, and a cap for sealably closing the open end of the liner. The breast milk-containing liner may thus be sealed by the cap and positioned in the rigid stand. The stand and capped liner may then be placed in a refrigerated environment so that the breast milk may be stored at cold temperatures and thereby preserved for future use.

Most preferably, the kits of the present invention, will also include a nipple cap for the bottle. The frozen breast milk in the flaccid liner may thus be defrosted and placed in the rigid tubular bottle so that the liner is dependently supported thereby. The nipple cap may then be threadably coupled to the bottle to allow the breast milk contents in the liner to be fed to the nursing infant.

These and other aspects and advantages will become more apparent after careful consideration is given to the following detailed description of the preferred exemplary embodiments thereof.

### BRIEF DESCRIPTION OF THE ACCOMPANYING DRAWINGS

Reference will hereinafter be made to the accompanying drawings, wherein like reference numerals throughout the various FIGURES denote like structural elements, and wherein;

FIG. 1 is a perspective view of an especially preferred embodiment of a kit in accordance with the present invention;

FIG. 2 is a perspective view of a preferred cap that may be employed to sealably close the open-ended flaccid liner employed in the kits of the present invention;

FIG. 3 is a ton plan view of the can depicted in FIG. 2;

FIG. 4 is a cross-sectional elevational view of the cap depicted in FIG. 2 as taken along line 4-4 in FIG. 3;

FIGS. 5A through 5H depict the steps by which the individual component parts in the kit depicted in FIG. 1 may be employed by the end user for the be collection, storage and/or feeding to a nursing infant; and

FIGS. 6A through 6C depict an alternate technique by which a filled liner may be capped in accordance with the present invention.

### DETAILED DESCRIPTION OF THE INVENTION

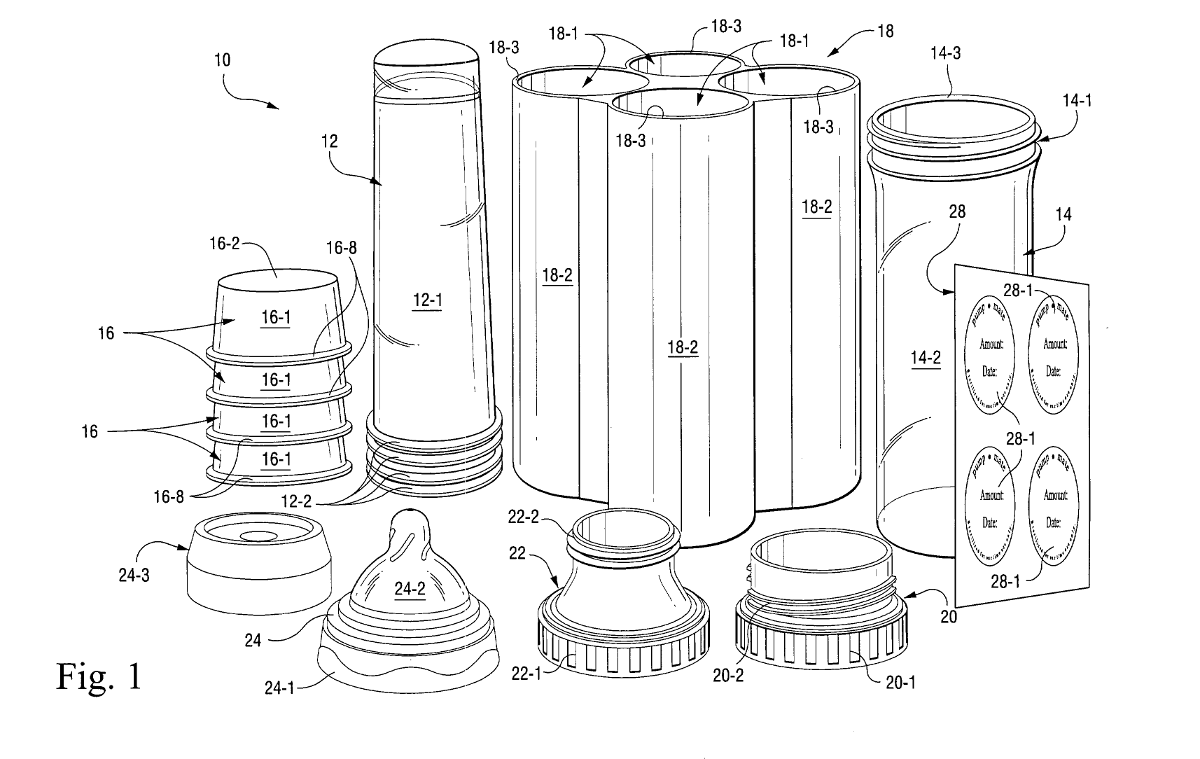

The individual components of an especially preferred kit 10 in accordance with the present invention is shown in accompanying FIG. 1. In this regard, the kit 10 of the present invention will necessarily include a number of flexible liners 12 each of which unitarily includes a flaccid elongate body portion 12-1 having a closed bottom, and an open-ended top defined by a self-supporting and shape-retaining annular flange member 12-2. Most preferably, the flexible liners 12 may be those described more fully in U.S. Pat. Nos. 4,836,764 and 5,091,231, the entire content of each being incorporated hereinto by reference.

By the term “self-supporting” is meant that the structure is capable of supporting its own weight against gravity without deformation. Thus, the flaccid body portion 12-1 is non-self-supporting since it is incapable of supporting its own weight against gravity. The term “shape retaining” means that the structure is capable of retaining and/or resiliently returning to its original shape after the application of a deformation force. Thus, the flange member 12-2 of the liner is yieldable to a deformation force, but is sufficiently pliant and resilient to return substantially to its originally annular shape after the deformation force is released.

The individual liners 12 are each adapted to be received by a generally tubular rigid bottle member 14 having a threaded upper end portion 14-1, an open-bottomed generally cylindrical sleeve portion 14-2 therebelow, and an annular upper rim 14-3 which defines an open top end. Each of the flaccid body portions 12-1 of the flexible liners 12 is therefore individually adapted to being inserted into the generally cylindrical interior region of the sleeve 14-2 so that the self-supporting flange member 12-2 thereof rests upon the annular upper rim 14-3. The weight of the breast milk contents within the body portion 12-1 of the liner 12 is thus dependently supported by the flange member 12-2 bearing against the rim 14-3.

The open-ended liners 12 may be sealably closed by a respective one of the caps 16. Most preferably, the same number of liners 12 and caps 16 are provided in each kit 10. Furthermore, the particular number of liners 12 and caps 16 is not critical to the functioning of the present invention and thus such number may be selected by the manufacturer. Although four such liners and caps 12, 16, respectively, are depicted in FIG. 1, such a number is for illustration only and does not limit the present invention in any way.

The liners 12 are also adapted to be inserted into a respective cylindrical receptacle 18-1 of a rigid stand 18. Each receptacle 18-1 is defined by a rigid generally cylindrical liner tube 18-2. As shown, a number of such liner tubes 18-2 may be integrally joined one to another to form a desired array. Again, the particular number of liner tubes 18-2 is not critical to the present invention, so that greater/lesser number of the same may be provided as compared to that shown in FIG. 1. Similar to the functional relationship between the annular flange 12-2 of the liner and the rim 14-3 of the bottle 14, the annular flanges 12-2 are adapted to rest upon, and thus be supported by, a respective one of the upper annular rims 18-3 of the liner tubes 18-2. In such a manner, individual liners 12 containing breast milk and sealably closed by means of a cap 16 may be positioned within, and supported by, a respective one of the liner tubes 18-2 of the stand 18. The stand 18 may thus be placed into a refrigerated environment so as to preserve the breast milk contents within the liner 12 held thereby.

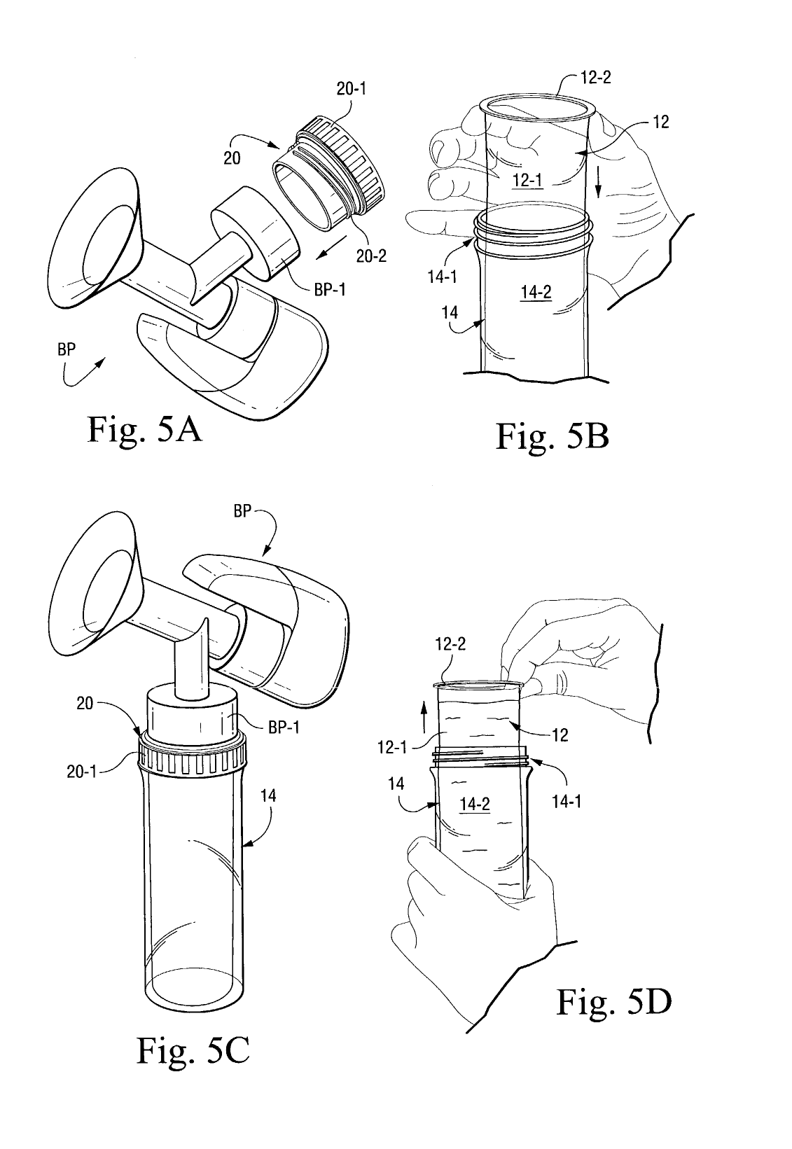

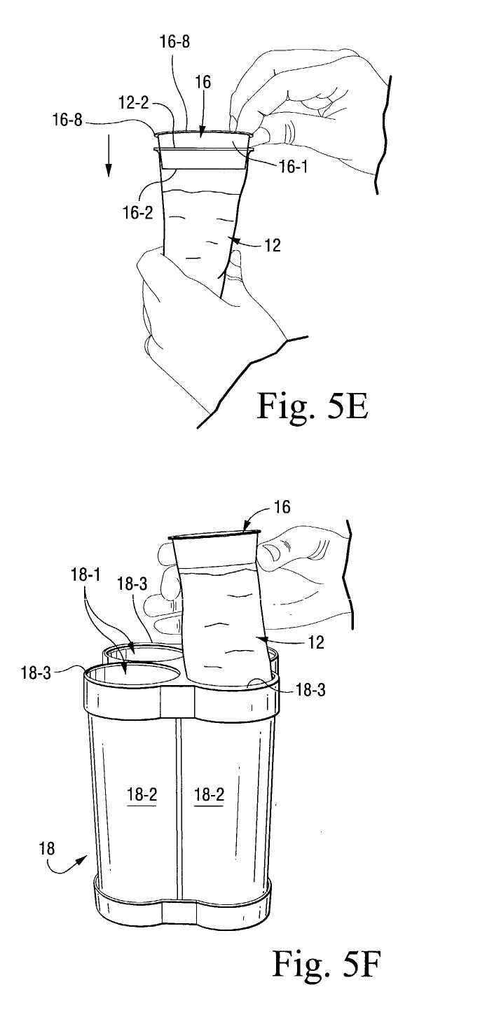

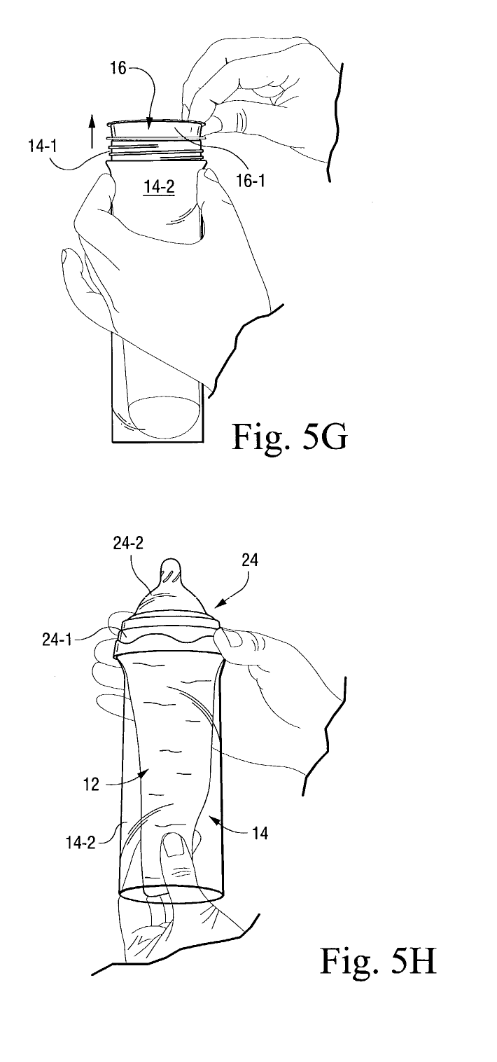

In order to assist the nursing mother, the kit 10 may contain breast pump adapters 20, 22. The adapters are provided with a lower threaded skirt portion 20-1, 22-1 which is adapted to being threadably coupled to the threaded upper portion 14-1 of the bottle 14. An upper threaded neck portion 20-2, 22-2 is in turn adapted to being threadably coupled to a commercially available breast pump BP (not shown in FIG. 1, but see FIG. 5A) in the nursing mother's possession. The adapters 20, 22 therefore allow the nursing mother to express breast milk directly into an empty one of the liners 12 dependently supported within the bottle 14. Most preferably, the adapters 20, 22 are those described more fully in copending and commonly owned U.S. Design patent application Ser. Nos. 29/171,415 and 29/171,416 each filed on Nov. 22, 2002 (the entire content of each prior-filed application being expressly incorporated hereinto by reference).

A nipple cap 24 is also most preferably provided as a part of the kit 10. The nipple cap 24 is, in and of itself, conventional. That is, the nipple cap 24 comprises a threaded lower skirt portion 24-1 adapted to being threadably engaged to the threaded upper portion 14-1 of the bottle 14. A pliant nipple 24-2 upwardly extends from the threaded skirt portion 24-1 and allows a nursing infant to receive breast milk therethrough from a liner 12 supported within the bottle 14. A snap-fit cover 24-3 for the nipple 24-2 may also be provided as part of the kit 10 so as to promote and maintain its cleanliness during use.

Advantageously, the kit 10 will also include a sheet 28 having a number of removable adhesively-backed label stickers 28-1 upon which information pertaining to the breast milk collection, e.g., the date and time of collection, may be marked. Each label sticker 28-1 is sized and configured so as to be affixed to the upper surface of the bottom wall 16-2 of the cap 16 as depicted in phantom line in FIG. 3. Most preferably, the number of individual labels 28-1 will be the same as the number of liners 12 and caps 16 provided in each kit 10. However, the particular number of labels 28-1, as with the particular number of liners 12 and caps 16, is not critical to the functioning of the present invention and thus such number may be selected by the manufacturer.

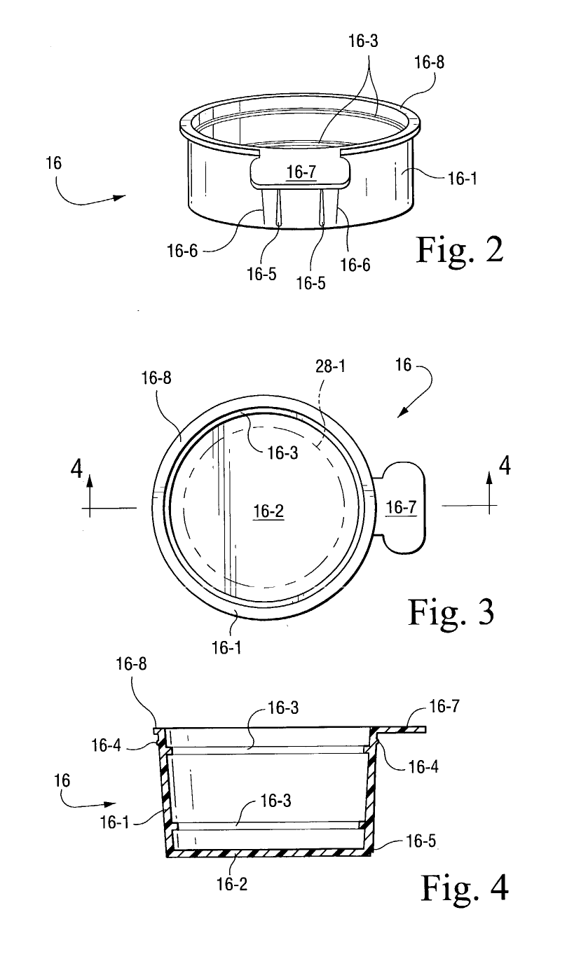

An exemplary cap 16 is depicted in accompanying FIGS. 2-4 as being generally cup-shaped and including a tapered side wall 16-1 terminating in a flat bottom wall 16-2. A pair of interior annular support ribs 16-3 are provided on an interior region of the side wall 16-1. An annular seal lip 16-4 is provided near an upper end of the side wall 16-1 so as to form a liquid-tight seal with the upper end of the liner 12 when positioned therewithin. The exterior surface of the side wall 16-1 is provided with a pair of vent ribs 16-5 which allow an amount of air within the liner 12 to be vented thereby permitting the cap 16 to be seated therewithin. A pair of score lines 16-6 are also provided in the side wall 16-1 so as to cooperate with a tear tab 16-7. By applying generally upward pressure on the tear tab 16-7 will cause the side wall to be torn through the annular rim 16-8 and along the score lines 16-6 thereby allowing the cap to be removed from the liner with greater ease.

The manner in which the various components within the kit 10 may be used is depicted schematically in accompanying FIGS. 5A-5H. A nursing mother should of course thoroughly wash her hands and breasts before handling and use of the components within the kit 10. In this regard, the components within the kit 10, especially the liners 12 and caps 16, may be pre-sterilized before being packaged in kit form and supplied to the end user.

In order to express breast milk, the nursing mother will select one of the supplied breast pump adapters 20 or 22 and attach it to the discharge port BP-1 of the breast pump BP as shown in FIG. 5A. In this regard, the adapter 20 is depicted in FIG. 5A for illustration purpose only, it therefore being understood that the discussion which follows is equally applicable to the use of adapter 22. A nursing mother may then insert a pre-sterilized empty liner 12 within the bottle 14 while avoiding contact with the liner's interior as shown in FIG. 5B. The bottle 14 with the empty liner 12 positioned therein may then be threadably engaged with the threaded skirt 20-1 of the adapter 20 as shown in FIG. 5C. The nursing mother may then proceed to express milk via the discharge port BP-1 of the breast pump BP directly into the liner 12 within the bottle 14.

When pumping is finished, one of the caps 16 may be marked with the date/time of the breast milk collection which, as noted previously, is most advantageously accomplished by marking such information on one of the label stickers 28-1 supplied with the kit 10, and then affixing the label sticker 28-1 onto the upper surface of the bottom wall 16-2 of cap 16 (e.g., in a position as shown in phantom line in FIG. 3). The bottle 14 may be threadably removed from the adapter 20 so as to gain access to the liner 12 now containing a quantity (e.g., between about 5 to 6 ounces) of freshly expressed breast milk. For ease of reference, the breast-milk containing liner 12 will be referred to hereinafter as a “partially filled” liner, since an amount of air will typically be present between the flange 12-2 and the liquid level within the body portion 12-1. The partially filled liner 12 may thus be removed carefully from the bottle 14 as shown in FIG. 5D. Once removed, the flaccid body portion 12-1 of the liner 12 is gently squeezed so as to discharge an amount of air therefrom as shown in FIG. 5E and thereby allow the date/time marked cap 16 to be sealably inserted into the liner's open end.

The capped liner 12 may then be positioned within a receptacle 18-1 of a respective liner tubes 18-2 as shown in FIG. 5F. The entire stand 18 with the partially filled liner 12 held thereby may thus be placed directly into a refrigerated environment (e.g., a refrigerator, freezer, cooler or the like) so as to preserve the breast milk contents. The process steps described above may be repeated to accumulate additional partially filled and capped liners 12 which may be placed into respective liner tubes 18-2 and then frozen.

When it is desired to feed the breast milk contents of a nursing infant, a partially filled and capped liner 12 containing chilled (or perhaps frozen) breast milk may be removed from the stand 18 in the refrigerated environment and warmed to essentially ambient room temperature. In this connection, the partially filled and capped liner 12 may be thawed, if frozen, in the refrigerator overnight and thereafter placed in warm water until the breast milk reaches room temperature. The partially filled liner should not, however, be warmed in a microwave oven as that may damage the liner and/or cause the breast milk contents thereof to become overheated to an extent that burn injury to the nursing mother and/or infant may result.

The warmed liner 12 may then be placed within the bottle 14 and the cap 16 removed therefrom as shown in FIG. 5G and discarded. The nipple cap 24 may then be threadably coupled to the threaded upper portion 14-1 of the bottle 14. The nipple-capped bottle 14 should be gently swirled or shaken so as to mix the breast milk contained within the liner as it may have separated as a result of its refrigerated storage. As shown in FIG. 5H, air may be removed from the liner 12 by gently squeezing the bottom of the liner 12 until a small amount of breast milk is discharged through the nipple 24-2. This will also allow the temperature of the breast milk to be tested. Thereafter, the nursing infant may be fed the breast milk contents within the liner 12. When not in use, the pliant nipple 24-2 may be protected by means of the nipple cover 24-3.

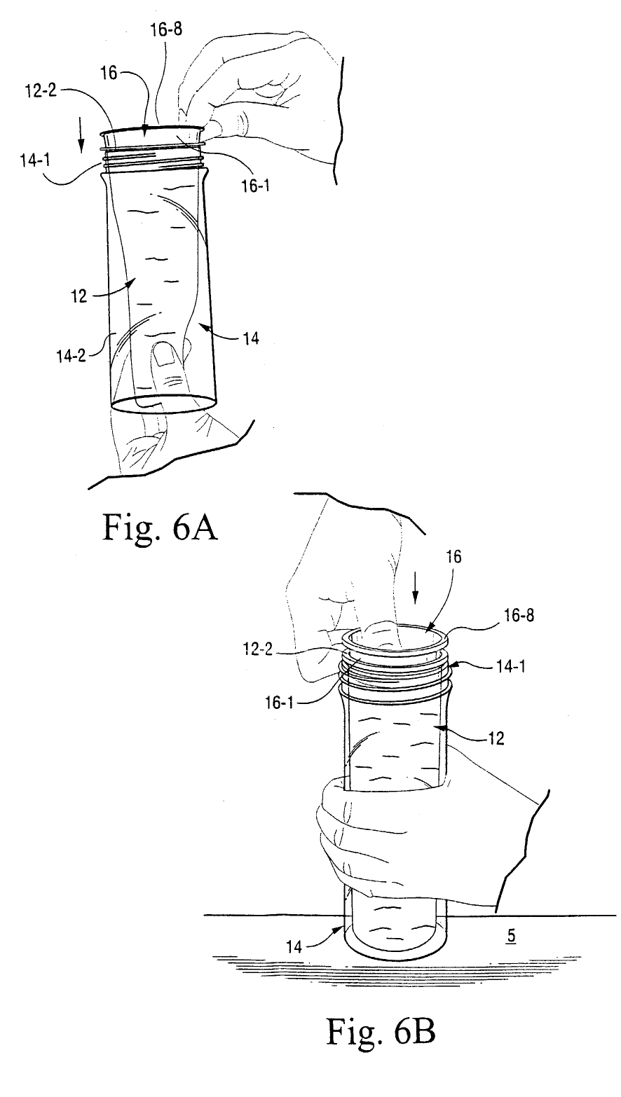

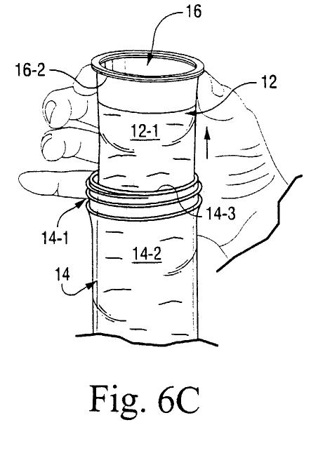

An alternative technique for capping a partially filled liner 12 is depicted in accompanying FIGS. 6A through 6C. In this regard, the technique described below starts with a partially filled liner 12 being seated within the interior of the bottle 14 such that the flange member 12-2 is dependently supported by the rim 14-3 thereof. That is, the partially filled liner 12 and bottle 14 will be removed as a unit from the breast pump BP following collection of the breast milk in a manner as described above. While in such a state, as shown in FIG. 6A, an amount of air may be expelled from the partially filled liner 12 by gently squeezing the flaccid body member 12-1 while simultaneously initially seating a respective one of the caps 16 (appropriately marked with one of the label stickers 28-1, if desired) within the open end of the liner 12. Expelling an amount of air from the liner 12 in such a manner will allow the cap 16 to be more easily seated since the trapped air within the liner 12 will not exert pressure thereagainst.

Once the cap 16 has been initially seated in the liner 12, the bottle may be placed on a firm surface S, for example, a tabletop, countertop, or the like, so the more firm force may be applied to the bottom wall 16-2 of the cap (e.g., by using one's thumb as shown in FIG. 6B). A soft “click” or tactile feel may be experienced when the cap 16 is fully seated due to the interaction of the seal lip 16-4 as it is pushed under the self-supporting annular flange 12-2 thereby establishing a liquid-tight seal with the liner 12. The sealed and capped partially filled liner 12 may then be removed from the bottle 14 as shown in FIG. 6C, and transferred to a receptacle 18-1 of the holder 18 as described above with reference to FIG. 5F.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A kit for the collection, storage and/or feeding of breast milk comprising:
a plurality of open-ended flexible liners for containing breast milk;
an outer rigid tubular bottle having a threaded upper portion for receiving a respective one of the flexible liners; and
at least one breast pump adapter having a lower threaded skirt for threaded engagement with the upper portion of the bottle with the liner received therewithin and an upper threaded neck to allow threaded engagement with a discharge port of a breast pump.

2. A kit as in claim 1, further comprising a plurality of seal caps for sealably closing the open end of the flexible liners.

3. A kit as in claim 2, wherein the seal caps include at least one vent rib to allow venting of air from the liner.

4. A kit as in claim 2 or 3, wherein the seal caps include a tear tab.

5. A kit as in claim 4, wherein the seal caps include a score line formed in a side wall of the cap and cooperating with the tear tab to allow the side wall to be torn by application of pressure to the tear tab so as to facilitate removal of the cap from the liner.

6. A kit as in claim 5, wherein the seal caps include a tapered side wall, and an annular seal rib formed on an upper exterior portion of said side wall for sealing engagement with a corresponding portion of said liner.

7. A kit as in claim 5, further comprising a stand comprised of plural rigid liner tubes each defining a cylindrical receptacle for receiving the liner therewithin.

8. A kit as in claim 2 or 3, wherein the seal caps include a tapered side wall, and an annular seal rib formed on an upper exterior portion of said side wall for sealing engagement with a corresponding portion of said liner.

9. A kit as in claim 1, 2 or 3, further comprising a stand comprised of plural rigid liner tubes each defining a cylindrical receptacle for receiving the liner therewithin.

10. A kit as in claim 9, further comprising a nipple cap having a threaded skirt portion adapted for threaded engagement with the threaded upper portion of the bottle, and a pliant nipple.

11. A kit as in claim 1, wherein each of said liners unitarily includes a flaccid elongate body portion, and an upper annular self-supporting flange surrounding said open end.

12. A kit as in claim 11, further comprising a plurality of seal caps for sealably closing the open end of the flexible liners.

13. A kit as in claim 12, wherein the seal caps include at least one vent rib to allow venting of air from the liner.

14. A kit as in claim 12 or 13, wherein the seal caps include a tear tab.

15. A kit as in claim 14, wherein the seal caps include a tapered side wall, and an annular seal rib formed on an upper exterior portion of said side wall for sealing engagement with a corresponding portion of said liner.

16. A kit as in claim 2, further comprising a plurality of labels to allow marking of respective ones of the seal caps.

17. A kit as in claim 1, further comprising a nipple cap threadably engageable with said bottle to allow breast milk contents to be fed to a nursing infant.

18. A kit as in claim 17, further comprising a cover for said nipple cap.

19. A kit for the collection, storage and/or feeding of breast milk comprising:
a plurality of open-ended flexible liners for containing breast milk;
a plurality of seal caps for sealably closing the open end of the flexible liners; and
a stand comprised of plural rigid liner tubes each defining a cylindrical receptacle for receiving the liner therewith in.

20. A kit as in claim 19, wherein the seal caps include at least one vent rib to allow venting of air from the liner.

21. A kit as in claim 19 or 20, wherein the seal caps include a tear tab.

22. A kit as in claim 21, wherein the seal caps include a score line formed in a side wall of the cap and cooperating with the tear tab to allow the side wall to be torn by application of pressure to the tear tab so as to facilitate removal of the cap from the liner.

23. A kit as in claim 21, wherein the seal caps include a tapered side wall, and an annular seal rib formed on an upper exterior portion of said side wall for sealing engagement with a corresponding portion of said liner.

24. A kit as in claim 19 or 21, wherein the seal caps include a tapered side wall, and an annular seal rib formed on an upper exterior portion of said side wall for sealing engagement with a corresponding portion of said liner.

25. A kit as in claim 19, further comprising at least one breast pump adapter having a lower threaded skirt for threaded engagement with the upper portion of the bottle with the liner received therewithin and an upper threaded neck to allow threaded engagement with a discharge port of a breast pump.

26. A kit as in claim 19, further comprising a nipple cap having a threaded skirt portion adapted for threaded engagement with the threaded upper portion of the bottle, and a pliant nipple.

27. A kit as in claim 26, further comprising a cover for said nipple cap.

28. A kit as in claim 19, further comprising a plurality of labels to allow marking of respective ones of the seal caps.

29. A kit for the collection, storage and/or feeding of breast milk comprising:
a plurality of open-ended flexible liners for containing breast milk;
an outer rigid tubular bottle having a threaded upper portion for receiving a respective one of the flexible liners;
a plurality of seal caps for sealably closing the open end of the flexible liners;
at least one breast pump adapter having a lower threaded skirt for threaded engagement with the upper portion of the bottle with the liner received therewithin and an upper threaded neck to allow threaded engagement with a discharge port of a breast pump;
a nipple cap having a threaded skirt portion adapted for threaded engagement with the threaded upper portion of the bottle, and a pliant nipple; and
a stand comprised of plural rigid liner tubes each defining a cylindrical receptacle for receiving the liner therewith in.

30. A kit as in claim 29, wherein the seal caps include at least one vent rib to allow venting of air from the liner.

31. A kit as in claim 29 or 30, wherein the seal caps include a tear tab.

32. A kit as in claim 31, wherein the seal caps include a score line formed in a side wall of the cap and cooperating with the tear tab to allow the side wall to be torn by application of pressure to the tear tab so as to facilitate removal of the cap from the liner.

33. A kit as in claim 32, wherein the seal caps include a tapered side wall, and an annular seal rib formed on an upper exterior portion of said side wall for sealing engagement with a corresponding portion of said liner.

34. A kit as in claim 29, wherein the seal caps include a tapered side wall, and an annular seal rib formed on an upper exterior portion of said side wall for sealing engagement with a corresponding portion of said liner.

35. A kit as in claim 29, further comprising a cover for said nipple cap.

36. A kit as in claim 29, further comprising a plurality of labels to allow marking of respective ones of the seal caps.

37. A method for the collection, storage and/or feeding of breast milk comprising:
(a) positioning an open-ended flexible liner within a rigid tubular bottle so that the liner is dependently supported by an upper rim of the bottle;
(b) threadably connecting an upper end of the bottle to a breast pump;
(c) expressing breast milk via the breast pump into the flexible liner so as to at least partially fill the same;
(d) removing the partially filled liner from the bottle; and
(e) sealably closing the open end of the liner with a removable seal cap.

38. The method of claim 37, further comprising:
(f) inserting the partially filled and sealably closed liner into a generally cylindrical receptacle of a rigid stand; and
(g) placing the stand into a refrigerated environment for storage.

39. The method of claim 38, further comprising:
(h) removing the liner from the stand and warming the breast milk therein;
(i) positioning the flexible liner with the warmed breast milk therein within the rigid tubular bottle so that the liner is dependently supported by the upper bottle rim; and
(j) threadably connecting a nipple cap to the upper portion of the bottle to allow the breast milk to be fed to a nursing infant.

40. The method of claim 37, further comprising (f) removing the cap from the liner, and (g) threadably connecting a nipple cap to the upper portion of the bottle to allow the breast milk to be fed to a nursing infant.

41. The method of claim 40, wherein step (f) comprises: (f1) applying pressure to a tear tab associated with the cap so as to tear a side wall thereof and thereby facilitate removal of the cap from the liner.

42. The method of claim 41, wherein step (f1) includes providing a score line in the cap side wall in cooperation with the tear tab.

43. The method of claim 37, further comprising (f) marking the seal cap with information pertaining to the breast milk collection.

44. The method of claim 43, wherein step (f) comprises marking information pertaining to the breast milk collection onto a sticker label, and affixing the sticker label onto the seal cap.

45. The combination comprising:
a flexible liner which includes a flaccid, non-self-supporting, elongate body portion having an open top and a closed bottom, and an annular self-supporting flange member bounding said open top; and
a seal cap seated within said open top so as to seal the open top against fluid leakage.

46. The combination as in claim 45, wherein said body portion and said flange member are unitary with one another.

47. The combination as in claim 45, wherein the seal cap includes at least one vent rib to allow venting of air from the liner.

48. The combination as in claim 45, 46 or 47, wherein the seal cap includes a tear tab.

49. The combination as in claim 48, wherein the seal cap includes a score line formed in a side wall of the cap and cooperating with the tear tab to allow the side wall to be torn by application of pressure to the tear tab so as to facilitate removal of the cap from the liner.

50. The combination as in claim 49, wherein the seal cap includes a tapered side wall, and an annular seal rib formed on an upper exterior portion of said side wall for sealing engagement with a corresponding portion of said liner.

51. The combination as in claim 45, wherein the seal cap includes a tapered side wall, and an annular seal rib formed on an upper exterior portion of said side wall for sealing engagement with a corresponding portion of said liner.

52. The combination as in claim 45, further comprising a label affixed to the seal cap.
